(19) United States
(12) Patent Application Publication
(10) Pub. No.: US 2009/0170147 A1
(43) Pub. Date: Jul. 2, 2009

(54) Forensic test kit and method for the detection of semen

(75) Inventor: Stephen Patrick Ashburn, Amarillo, TX (US)

Correspondence Address: STEPHEN P. ASHBURN, P.O. Box 300, 9 Custer Avenue, Fort Meade, SD, 57741, US

(73) Assignee: Stephen Patrick Ashburn, Amarillo, TX (US)

(21) Appl. No.: 11/964,566

(22) Filed: Dec. 26, 2007

(51) Int. Cl. C12Q 1/42

(52) U.S. Cl. 435/21

(57) ABSTRACT

The present invention relates to a forensic test kit and method for the detection of semen. This kit is comprised of two main components: acid phosphatase (AP) test strips and prostate specific antigen (PSA) test strips, which work together to provide evidence of semen on fabrics and other surfaces. The AP strips are used first to test an item, which provides presumptive evidence of semen, while follow-on testing by the more sensitive and specific PSA strips provides confirmatory evidence.

### FIELD OF THE INVENTION

The present invention relates to forensic chemistry. In particular, it relates to a combination of two analytical tests and method thereof for the detection of semen on fabrics and other surfaces.

### BACKGROUND OF THE INVENTION

Conservative statistics indicate that about 14% of women and 22% of men have had affairs sometime in their marriage [Laumann et al. “The Social Organization of Sexuality: Sexual Practices in the United States”; University of Chicago Press: Chicago, page 216, 1994]. According to a recent study by the Centers for Disease Control, about 4% of both married men and women had more than one sexual partner in the previous twelve months [Mosher et al. “Sexual Behavior and Selected Health Measures: Men and Women 15-44 Years of Age, United States, 2002,” Advance Data, 362, page 10, 2005]. This figure rises to 15% in the case of unmarried couples cohabiting. These data indicate that infidelity is a significant problem in the United States, and there exists a need to objectively test spouses for sexual activity. For women, one such test is for the presence of semen.

When a man has sexual intercourse with a woman, semen is deposited into the woman's vagina. Immediately after intercourse, most of the semen flows back out, but some is retained in the vagina and slowly is discharged over a period of several days [Hooft et al, Am. J. Forensic Med. Pathol., 18, pages 45-49, 1997]. Semen has over 900 identified proteins [Pilch et al, Genome Biology, 7(R40), 2006] among which are semenogelin I and II (gel-forming proteins produced by the seminal vesicles), prostate-specific antigen (a protease which breaks down semenogelin), and acid phosphatase (which breaks down spermatozoa cell membranes) [Tanaka et al, FEBS Lett., 571, pages 197-204, 2004]. These proteins can be identified by immunochromatographic assay, which forms the principle of the PSA test in the present invention. Acid phosphatase can be detected by the classic test first reported by Babson et al in Am.J.Clin.Pathol., 32, pages 88-91 (1959), which forms the principle of the AP test in the present invention. This test relies on the catalytic hydrolysis of 1-naphthyl phosphate to form 1-naphthol, which in turn reacts with an aryl diazonium salt, forming an intensely colored azo dyestuff. In addition to proteins, semen also has unusually high concentrations of zinc (100-200 mg/L v. 1 mg/L in plasma) [Owen et al, J.Androl., 26, pages 459-469, 2005]. Zinc (like AP and PSA) is produced by the prostate gland and after ejaculation, 50% is bound to seminal vesicle proteins. Zinc acts to stabilize DNA inside spermatozoa, is a cofactor in enzymatic reactions and also may catalyze the gel-forming reaction between semenogelin I and II. Semen may be detected by the modified zinc test of Hooft and van de Voorde [Hooft et al, Forensic Sci. Int., 53, pages 131-133, 1992].

The semen flowing back out of a woman's vagina (“backflow”) is deposited on her underwear or absorbent pad. These items conveniently can be tested with the semen detection kit described in this invention. The kit also can be used to test stains on other fabrics and surfaces.

All stains on women's undergarments are not semen. In fact, asymptomatic women roduce, on the average, 1.5 g of vaginal fluid per day, which typically leaves a white-to-beige stain [Beckmann et al. “Obstetrics and Gynecology, Second Edition”; Williams & Wilkins: Baltimore, page 294, 1995]. Semen stains, on the other hand are white and appear mainly just after intercourse. The next day, discharge of residual semen may not be visible at all. Thus, it is impossible to tell visually whether a suspicious stain is semen, and men must rely on analytical methods of detection such as that described in the present invention.

Other methods for semen detection have been described. Immunochromatographic test strips for PSA, first described by Yoshiki [An et al, Cancer Lett., 162, pages 135-9, 2001] are commercially available from several suppliers and have been validated for use in forensic investigations [Laux et al, online, retrieved 2008]. A similar test for semenogelin recently has been described [Pang et al, Forensic Sci. Int., 169, pages 27-31, 2007]. Test methods for acid phosphatase (AP) also have been described, for example as a test strip in U.S. Pat. No. 5,981,206 (Arter et al) and as a solution in U.S. Pat. No. 3,002,893 (Babson) and U.S. Pat. No. 6,764,856 (Holmes et al).

Recently Hooft et al showed that the modified zinc test was more sensitive and specific for the detection of semen than the classic acid phosphatase test [Hooft et al, Am. J. Forensic Med. Pathol., 18, pages 45-49, 1997], although the ready availability of AP test strips may be a reason why zinc strips were not more widely adopted.

Although PSA and AP tests for semen have been described, the combination of both tests being used synergistically as a detection kit and method has not. Furthermore, it is not intuitively obvious that the use of both test methods would increase both sensitivity and specificity for the detection of semen. In addition, it is not obvious that concurrent use of both tests would provide a convenient source of both presumptive and confirmatory evidence for semen in forensic investigations.

### SUMMARY OF THE INVENTION

The present invention comprises a dual-action test kit and method consisting primarily of AP test strips and PSA test strips, which work together to provide evidence of semen on garments and other items. The AP strips are used first to test an item, which provides presumptive evidence of semen, while follow-on testing by the more sensitive and specific PSA strips provides confirmatory evidence. This method provides for 100% sensitivity and 100% specificity for semen when a suspect garment is tested less than 12 hours after intercourse.

The kit is designed to be easy to use and yields instant results with the AP test strips, or results after 10 minutes with the more sensitive PSA test. It is designed to detect traces of semen on a woman's undergarment which has been discharged after sexual intercourse, and up to 36 hours later.

The AP strips can detect semen down to a 1/2000 dilution, while the PSA strips can detect semen to a 1/1,000,000 dilution. The kit is designed to be used by men who suspect their spouse may be engaged in sexual activity outside of their relationship. It also can be used by professional investigators, and parents concerned about whether their teenage daughters are sexually active.

The preferred method for the detection of semen on fabrics is to wet the suspect area with a few drops of water, and then press an AP strip against it. A color change within 15 seconds to bright purple is a POSITIVE test. If the test is POSITIVE, a PSA test is carried out to confirm the presence of semen. If the test is NEGATIVE, but there exists a high degree of suspicion that there may be a trace of semen on the garment, a PSA test is performed anyway. The PSA test is performed by extracting the suspect area of the item in a coffee cup with a minimal amount of water, followed by wringing out the item, and placing a PSA test strip into the solution. A POSITIVE test is indicated by the presence of a visible line in the test area of the strip. If the test is POSITIVE, the presence of semen is confirmed.

The AP test strip assemblies are prepared by adsorbing reagent onto filter paper, drying, cutting into strips and mounting to a plastic backing.

### BRIEF DESCRIPTION OF THE DRAWING

The present invention is described in detail below with reference to the following drawing.

FIG. 1 simplified diagram for performing a PSA test.

### DETAILED DESCRIPTION OF THE INVENTION

The present invention relates to a detection kit and method for the identification of semen in suspicious stains on garments and other items. The kit is comprised of two main components: AP test strips and PSA test strips, which work synergistically to determine the presence of semen with a higher degree of sensitivity and specificity than either test used alone.

A preferred embodiment of the kit contains AP strips in a sealed canister with a desiccant cap, PSA strips in individual sealed pouches, and a dropper. A specific example is given below:

Materials Supplied in the Kit
15 AP test strips in a sealed canister with a desiccant cap
10 PSA test strips in individual sealed pouches
5-mL dropper
Instruction sheet

Materials Not Supplied but Required
Distilled or deionized water
Latex gloves (available from drug store)
Coffee cup

In this example, the AP strips are stored in a tightly sealed canister and PSA strips are kept in sealed individual pouches containing a desiccant packet. This embodiment protects the strips from air and moisture until use. According to the preferred method, the user is required to supply distilled or deionized water, latex gloves and a coffee cup. The purified water ensures the absence of contaminants such as chlorine which might affect the outcome of the test, and gloves protect the investigator from any infectious organisms which may be present on the item. The coffee cup provides a convenient extraction vessel for performing a PSA test.

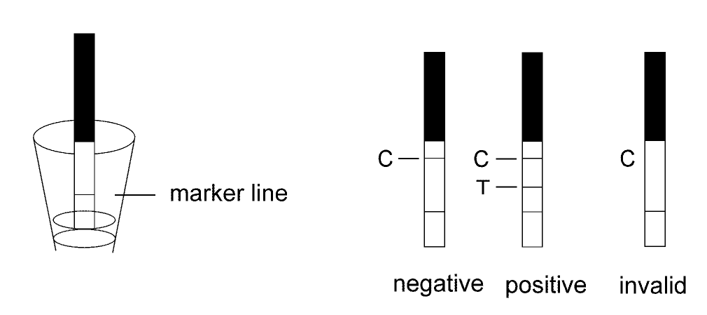

The preferred method for analyzing fabrics is to wet the suspect area with 2-5 drops of water, and then press an AP strip against it. A color change within 15 seconds to bight purple is a POSITIVE test. If the test is POSITIVE, a PSA test is carried out. If the test is NEGATIVE, but suspicion exists that there may be a trace of semen on the item, a PSA test is performed anyway. The PSA test is performed by extracting the suspect area of the garment or other item in a coffee cup with water, and then placing a PSA test strip into the solution. A POSITIVE test is indicated by the presence of a visible line in the test area of the strip. A simplified diagram for conducting a PSA test is shown in FIG. 1. A specific example of the preferred method is given below:

Simple Procedure
1. AP test. Place 2-5 drops of distilled or deionized water on a suspect area of the garment. Press an AP strip against it. A color change within 15 seconds to bright purple is a POSITIVE test. If the test is POSITIVE, proceed with a PSA test to confirm the presence of semen. If the test is NEGATIVE, but you are suspicious there might be a trace of semen on the garment, do the PSA test anyway.
   Alternative procedure: place a cotton-tipped swab against the wetted area of the garment, and then press the cotton-tipped swab against an AP test strip. This procedure will avoid leaving any stains on the garment.
2. PSA test. Place 15 mL of water in a coffee cup using the supplied dropper. Then, manually extract the suspect area (i.e. crotch) of the garment by repeatedly allowing water to soak in, then pressing it out. Finally, wring out the garment into the cup. Place a PSA test strip into the cup and wait 10 minutes. (It may be necessary to tilt the cup on edge to immerse the strip. Do not immerse the strip above the marker line.) Then, take the test strip out and lay it on a clean dry surface. Read the test strip after 10 minutes. A POSITIVE test is indicated by two lines as shown in FIG. 1. A strongly positive test will be clearly visible within two minutes, while a weakly positive test may take the entire 20 minutes to become evident.
   If you are testing absorptive pads (used during a woman's menstrual period), then place 25 mL of water into the coffee cup (for a full pad) or 10 mL for a mini-pad, and repeatedly extract the pad manually. Then, wring out the pad into the cup and discard it. Do the PSA test as usual.
   If you are testing a stain on a surface (for example, a car seat), then wipe the stain with a small piece of wet cloth and manually extract it in a coffee cup using a minimal amount of water. Do the PSA test as usual.
   NOTE: latex gloves are recommended for these procedures.

The AP test strip assemblies are prepared according to a modification of the method of Babson [Babson et al, Am.J.Clin.Path., 32, pages 88-91, 1959]. In the preferred embodiment, Whatman Grade 1 Qualitative Standard Filter Paper is used to absorb a solution of citrate buffer (prepared from equimolar amounts of citric acid and trisodium citrate), 1-naphthyl phosphate disodium salt and Dye Fast Blue B Salt, then dried and cut into strips of approximately 15 mm by 40 mm. The solution is prepared so that each strip contains approximately 5 mg (22 μmol) of citrate buffer, 1 mg (3.7 μmol) of 1-naphthyl phosphate and 1.6 mg (3.4 μmol) of Dye Fast Blue B Salt. The strips then are mounted to a plastic backing of approximately 15 mm by 60 mm, and stored in a canister with a desiccant cap. It should be noted that the reagents used to prepare the strips, as well as the strips themselves are hygroscopic and should be protected from moisture. The preferred embodiment affords users a convenient source of highly sensitive test strips for detecting acid phosphatase, and thereby semen. It is not intuitively obvious from the literature what concentration of reagents to use, or what filter paper or backing material is required to make the most sensitive test strip. The preferred method consists of holding the assembly by the plastic portion and using it to carry out a detection test for semen as described above. This scheme allows users to handle the test strips without touching the highly sensitive coated portion of the strip. The strips are validated using standard dilutions of semen, as well as testing on women's undergarments at intervals after intercourse, by which method their sensitivity can be determined. In a typical batch, the strips are found to have a detection limit of a 1/2000 dilution of semen. They typically can detect semen on women's undergarments which has been discharged up to 17 hours after intercourse.

PSA strips typically are obtained commercially as single immunochromatographic strips in a sealed pouch containing a desiccant packet. They are validated by testing against a series of semen dilutions, by which method their sensitivity can be determined. A typical batch of strips can detect semen to a dilution of 1/1,000,000, corresponding roughly to a PSA concentration of 4 ng/mL. At this concentration the strips have 100% sensitivity and 100% specificity for PSA [An et al, Cancer Lett., 162, pages 135-9, 2001], and by inference for semen.

The PSA strips are semi-quantitative, meaning that the control line is also an internal standard calibrated for a color intensity corresponding to a PSA concentration of 4 ng/mL. By comparing the color intensity of the test line against that of the control line, an investigator easily can see whether the PSA concentration is greater or less than 4 ng/mL. Since the specificity of the test is 100% at or above this concentration, the investigator can be certain that semen is present if the test line is equal or greater in intensity than the control line. A barely visible test line corresponds to a PSA concentration of about 2 ng/mL and has a specificity of 90% [An et al, Cancer Lett., 162, pages 135-9, 2001], meaning that the probability of a false positive result is 10%. Although PSA is present in small amounts in some bodily fluids of females, these small amounts have been shown not to interfere with the immunochromatographic detection of semen [Laux et al, online, retrieved 2008].

By means of the preferred method, the AP strips typically can detect semen on women's undergarments up to 17 hours after intercourse. Garments tested closer to the time of intercourse give a more strongly POSITIVE spot test. The intensity of the spot test generally decreases linearly with time.

When PSA strips are used to analyze garments by the preferred method, semen typically can be detected up to 36 hours after intercourse. The intensity of the test line typically decreases rapidly after 12 hours. This observation can be attributed to the rapid denaturization of seminal proteins by the acidic pH of the vagina, among other factors. A volunteer subject typically has semen visible in her vagina 36 hours after intercourse as a white, stringy substance; however, no marker proteins can be detected. Thus, seminal proteins appear to become denatured by the hostile chemical environment of the vagina, in a fashion similar to egg whites when they are cooked. Denaturization causes irreversible changes to the three-dimensional structure of proteins, which structure is critical for their immunochromatographic detection.

In case of a weak test, the use of both strips synergistically indicates the presence of semen with greater probability than either test taken by itself. For example, if a weakly positive PSA test has 90% specificity, then a weakly positive AP test additionally raises the specificity of the overall effort and provides the investigator with a more certain conclusion that the item being tested does in fact contain semen. While the invention has been described in detail with respect to the preferred embodiments thereof, it must be understood that changes can be made within the spirit and scope of the invention. All references cited herein, including patents, books, journal articles and other published prior art are incorporated for the purpose of teaching and understanding pertinent to this invention.

I claim:

1. A semen detection kit comprised of two main components: acid phosphatase (AP) test strips and prostate specific antigen (PSA) test strips.

2. A method for the detection of semen, in which an item is tested first with AP test strips to provide presumptive evidence of semen, followed by testing with the more sensitive and specific PSA strips for confirmatory evidence.

3. An AP test strip assembly consisting of an AP test strip bound to a plastic backing.
